(19) United States
(12) Patent Application Publication
(10) Pub. No.: US 2023/0190843 A1
(43) Pub. Date: Jun. 22, 2023

(54) METHODS FOR ENHANCING SLEEP QUALITY

(71) Applicant: Kitt Bio, Inc., Culver City, CA (US)

(72) Inventor: Andrew D. Herr, Pacific Palisades, CA (US)

(73) Assignee: Kitt Bio, Inc., Culver City, CA (US)

(21) Appl. No.: 17/898,017

(22) Filed: Aug. 29, 2022

Related U.S. Application Data
(60) Provisional application No. 63/238,398, filed on Aug. 30, 2021.

(51) Int. Cl. A61K 36/185; A61K 31/7048; A61K 45/06; A61P 25/20

(52) U.S. Cl. CPC A61K 36/185; A61K 31/7048; A61K 45/06; A61P 25/20

(57) ABSTRACT

Provided herein are compositions comprising a black currant berry product and methods of providing enhanced sleep quality by administration of the compositions.

### CROSS-REFERENCE TO RELATED PATENT APPLICATIONS

This application claims priority to U.S. Provisional Application 63/238,398 filed Aug. 30, 2021, the entire contents of which are incorporated herein by reference.

### FIELD

Described herein are methods for enhancing sleep quality using compositions comprising a black currant berry product as described herein, as well as black currant berry products and compositions comprising black currant berry products useful in such methods.

### SUMMARY

Provided herein, in one aspect, are methods of providing enhanced sleep quality in a human subject in need thereof, the methods comprising orally administering to the subject an effective amount of a composition comprising a black currant berry product. The enhanced sleep quality may comprise one or more selected from increased rapid eye movement (REM) sleep, increased deep non-REM sleep, shortened sleep latency, reduced unintended wake-ups, improved ability to fall back asleep, increased overall sleep time, and enhanced refreshed feeling upon wake-up. Also provided herein, in one aspect, are black currant berry products for use in methods of providing enhanced sleep quality in a human subject in need thereof, wherein the enhanced sleep quality may comprise one or more selected from increased rapid eye movement (REM) sleep, increased deep non-REM sleep, shortened sleep latency, reduced unintended wake-ups, improved ability to fall back asleep, increased overall sleep time, and enhanced refreshed feeling upon wake-up.

The black currant berry product may comprise black currant berry extract, black currant puree, black currant juice, black currant syrup, or a combination of two or more thereof. The black currant berry product may comprise one or more selected from anthocyanins and phenolic compounds. The black currant berry product may comprise anthocyanins in an amount from about 0.1% w/w to about 100% w/w based on the weight of the black currant berry product. The black currant berry product may comprise anthocyanins in an amount from about 20% w/w to about 50% w/w based on the weight of the black currant berry product. The black currant berry product may comprise one or more anthocyanins selected from delphinidin-3-glucoside, delphinidin-3-rutinoside, cyanidin-3-rutinoside, cyanidin-3-glucoside, cyanidin-3-O-glucoside, cyanidin-3-glucosylrutinoside, cyanidin-3-sophoroside, and peonidin-3-glucoside. The black currant berry product may comprise one or both of delphinidin-3-glucoside and delphinidin-3-rutinoside. In some embodiments, the black currant berry product comprises from about 30% w/w to about 50% w/w delphinidin-3-rutinoside, from about 4% w/w to about 25% w/w delphinidin-3-glucoside, from about 25% w/w to about 45% w/w cyanidin-3-rutinoside, and from about 2% w/w to about 10% w/w cyanidin-3-glucoside, all based on the total weight of anthocyanins present in the black currant berry product. In some embodiments, the black currant berry product comprises from about 1% w/w to about 99% w/w delphinidin-3-rutinoside and from about 99% w/w to about 1% w/w delphinidin-3-glucoside, based on the total weight of anthocyanins present in the black currant berry product, including about 10% delphinidin-3-rutinoside and about 90% w/w delphinidin-3-glucoside, about 25% delphinidin-3-rutinoside and about 75% w/w delphinidin-3-glucoside, about 33.3% delphinidin-3-rutinoside and about 66.6% w/w delphinidin-3-glucoside, about 50% delphinidin-3-rutinoside and about 50% w/w delphinidin-3-glucoside, about 66.6% delphinidin-3-rutinoside and about 33.3% w/w w/w delphinidin-3-glucoside, about 25% delphinidin-3-rutinoside and about 75% w/w delphinidin-3-glucoside, and about 90% delphinidin-3-rutinoside and about 10% w/w delphinidin-3-glucoside. In some embodiments, the black currant berry product further comprises one or more proanthocyanidin compounds.

The black currant berry product may be administered at a dose of from about 1 mg to about 5000 mg. The composition comprising the black currant berry product may be administered at a dose that provides an amount of black currant berry product equivalent to from about 10 mg to about 800 mg, including from about 10 mg to about 400 mg, of a black currant berry product having an anthocyanin content of about 35% w/w, based on the total weight of the black currant berry product. In some embodiments, the composition is administered at a dose that provides an amount of black currant berry product equivalent to from about 30 mg to about 400 mg, including from about 30 mg to about 200 mg, of a black currant berry product having an anthocyanin content of about 35% w/w, based on the total weight of the black currant berry product.

The composition may be a solid composition, optionally in the form of a capsule, tablet, or powder. The composition may be a liquid composition, optionally in the form of a liquid concentrate or beverage. The black currant berry product may be the sole physiologically active agent present in the composition.

In some embodiments, the method or use further comprises administering one or more additional physiologically active agents. In some embodiments, the one or more additional physiologically active agents are selected from L-theanine, glycine, melatonin, gamma-aminobutyric acid, magnesium L-threonate, and any combination of two or more thereof. In some embodiments, the method or use further comprises administering one or more of (i) from about 50 mg to about 800 mg L-theanine, (ii) from about 500 mg to about 8000 mg glycine, (iii) from about 0.1 mg to about 10 mg melatonin, (iv) from about 50 mg to about 2000 mg magnesium L-threonate, and (v) from about 100 mg to about 2000 mg gamma-aminobutyric acid. In some embodiments, the method or use further comprises administering one or more of (i) from about 50 mg to about 400 mg L-theanine, (ii) from about 500 mg to about 5000 mg glycine, (iii) from about 0.1 mg to about 10 mg melatonin, (iv) from about 50 mg to about 2000 mg magnesium L-threonate, and (v) from about 100 mg to about 2000 mg gamma-aminobutyric acid.

Also disclosed herein uses of the products and compositions described herein in the preparation of medicaments for providing enhanced sleep quality.

### DETAILED DESCRIPTION

Described herein are methods for enhancing sleep quality by orally administering a composition comprising a black currant berry product as described herein. As described in more detail below and illustrated in the example(s), the present inventor surprisingly found that oral administration of a composition comprising a black currant berry product as disclosed herein is effective to enhance sleep quality, such as one or more of increased rapid eye movement (REM) sleep, increased deep non-REM sleep, shortened sleep latency, reduced unintended wake-ups, improved ability to fall back asleep, increased overall sleep time, and enhanced refreshed feeling upon wake-up. Thus, also described herein are black currant berry products and compositions comprising black currant berry products useful in such methods.

Various embodiments are described herein. It should be noted that the specific embodiments described herein are not intended as an exhaustive description or as a limitation to the broader aspects discussed herein. To the extent an aspect is described in conjunction with a particular embodiment, it should be understood that the aspect is not necessarily limited to that embodiment, but can be practiced with any other embodiments.

Technical and scientific terms used herein have the meanings commonly understood by one of ordinary skill in the art to which the present invention pertains, unless otherwise defined. Reference is made herein to various methodologies known to those of ordinary skill in the art. Suitable materials and/or methods known to those of ordinary skill in the art can be utilized in carrying out the present invention based on the guidance provided herein. However, specific materials and methods are described for illustration. Materials, ingredients and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth. As used herein, “about” will be understood by persons of ordinary skill in the art and will vary to some extent on the context in which it is used. In general, the term “about” is intended to embrace up to plus or minus 10% of the particular term. For example, “about 10” is intended to embrace 9-11, and all values therebetween. The recitation of ranges of values herein are intended to serve as shorthand for each separate value falling within the range, unless otherwise indicated, and it should be understood that each separate value is disclosed as if it were individually expressly stated herein.

All methods described herein can be performed in any suitable order unless otherwise indicated herein or otherwise clearly contradicted by context. All examples and exemplary language (including “e.g.” and “such as”) provided herein are intended to better illuminate embodiments of the invention and do not impose limitations on the scope of the invention.

### Sleep and Sleep Quality

There are two phases of sleep: non-REM sleep and REM (rapid eye movement) sleep. Non-REM sleep occurs first and generally includes three stages. Stage 1 of non-REM sleep is the transition from wakefulness to sleep, during which heartbeat, breathing, and muscle activity generally slow down, although sudden muscle contractions may also occur at this time. During stage 2 of non-REM sleep, brain waves, heart rate, and body temperature generally decrease further, but with brief bursts of electrical activity in the brain. Stage 3 non-REM sleep is deep sleep (also referred to hereinafter as “deep non-REM sleep”), during which the brain produces delta waves and muscles are relaxed. During REM sleep, brain activity increases again and intense dreaming may be experienced. A human body cycles through non-REM sleep and REM sleep during a normal night of sleep, usually beginning with non-REM sleep.

Sleep quality may be assessed with reference to various factors, including duration of REM sleep, duration of deep non-REM sleep, measurement of sleep latency (i.e., length of time to accomplish transition from wakefulness to sleep, usually the first stage of non-REM sleep), number of unintended wake-ups, ability to fall back to sleep after wake-up, duration of overall sleep, and presence of refreshed feeling upon wake-up.

### Black Currant Berry Compositions

As noted above, described herein are methods for enhancing sleep quality by orally administering a composition comprising a black currant berry product, as well as black currant berry products and compositions comprising black currant berry products useful in such methods. In this context, the term “black currant berry product” identifies a product obtained from black currant berries, and is distinct from a product obtained from black currant seeds, such as black currant seed oil.

A black currant berry product as described herein may comprise a black currant berry extract, a black currant berry puree, a black currant berry juice, a black currant berry syrup, or a combination of any two or more thereof. As used herein, a “black currant berry extract” may be obtained through aqueous or organic extraction of black currant berries (or of a black currant berry puree, or other suitable preparation of black currant berries), including water extraction, ethanol extraction, or a combined water/ethanol extraction. As used herein, a “black currant berry extract” may be in a liquid or solid (e.g., powder) form.

The black currant berry product may be produced from berries of Ribes nigrum. Non-limiting examples of Ribes nigrum cultivars suitable for this purpose include Ben Ard, Ben Connan, Ben Lomond, Ben Alder, Ben Tirran, Ben Hope, Ben Sarek, Big Ben, Crusader, Consort, Ebony, Foxendown, and Titania.

The black currant berry product may comprise one or more anthocyanins present in black currant berries. Additionally or alternatively, the black currant berry product may comprise one or more non-anthocyanin phenolic compounds present in black currant berries. Additionally or alternatively, the black currant berry product may comprise one or more proanthocyanidin compounds present in black currant berries.

The black currant berry product may comprise one or more anthocyanins selected from delphinidin-3-glucoside, delphinidin-3-rutinoside, cyanidin-3-rutinoside, cyanidin-3-glucoside, cyanidin-3-O-glucoside, cyanidin-3-glucosylrutinoside, cyanidin-3-sophoroside, and peonidin-3-glucoside. The black currant berry product may comprise one or both of delphinidin-3-glucoside and delphinidin-3-rutinoside.

For example, the black currant berry product may comprise from about 30% w/w to about 50% w/w delphinidin-3-rutinoside, based on the total weight of anthocyanins present in the black currant berry product. This includes about 30%, 31%, 32%, 33%, 34%, 35%, 36%, 37%, 38%, 39%, 40%, 41%, 42%, 43%, 44%, 45%, 46%, 47%, 48%, 49% or 50% w/w, or any value therebetween, of delphinidin-3-rutinoside based on the total weight of anthocyanins present in the black currant berry product.

Additionally or alternatively, the black currant berry product may comprise from about 4% w/w to about 25% w/w delphinidin-3-glucoside, based on the total weight of anthocyanins present in the black currant berry product. This includes about 4%, 5%, 6%, 7%, 8%, 9%, 10%, 11%, 12%, 13%, 14%, 15%, 16%, 17%, 18%, 19%, 20%, 21%, 22%, 23%, 24%, or 25% w/w, or any value therebetween, of delphinidin-3-glucoside based on the total weight of anthocyanins present in the black currant berry product.

Additionally or alternatively, the black currant berry product may comprise from about 25% w/w to about 45% w/w cyanidin-3-rutinoside, based on the total weight of anthocyanins present in the black currant berry product. This includes about 25%, 26%, 27%, 28%, 29%, 30%, 31%, 32%, 33%, 34%, 35%, 36%, 37%, 38%, 39%, 40%, 41%, 42%, 43%, 44%, or 45% w/w, or any value therebetween, of cyanidin-3-rutinoside, based on the total weight of anthocyanins present in the black currant berry product.

Additionally or alternatively, the black currant berry product may comprise from about 2% w/w to about 10% w/w cyanidin-3-glucoside, based on the total weight of anthocyanins present in the black currant berry product. This includes about 2%. 3%, 4%, 5%, 6%, 7%, 8%, 9%, or 10% w/w, or any value therebetween, of cyanidin-3-glucoside, based on the total weight of anthocyanins present in the black currant berry product.

For example, the black currant berry product may comprise from about 30% w/w to about 50% w/w delphinidin-3-rutinoside, from about 4% w/w to about 25% w/w delphinidin-3-glucoside, from about 25% w/w to about 45% w/w cyanidin-3-rutinoside, and from about 2% w/w to about 10% w/w cyanidin-3-glucoside, all based on the total weight of anthocyanins present in the black currant berry product. In a specific example, the black currant berry product comprises about 40% w/w delphinidin-3-rutinoside, about 15% w/w delphinidin-3-glucoside, about 35% w/w cyanidin-3-rutinoside, and about 5% w/w cyanidin-3-glucoside, all , based on the total weight of anthocyanins present in the black currant berry product.

In some embodiments, the black currant berry product comprises delphinidin-3-rutinoside and delphinidin-3-glucoside. For example, a black currant berry product may comprise from about 1% w/w to about 99% w/w delphinidin-3-rutinoside and from about 99% w/w to about 1% w/w delphinidin-3-glucoside, based on the total weight of anthocyanins present in the black currant berry product, including about 10% delphinidin-3-rutinoside and about 90% w/w delphinidin glucoside, about 25% delphinidin-3-rutinoside and about 75% w/w delphinidin-3-glucoside, about 33.3% delphinidin-3-rutinoside and about 66.6% w/w delphinidin-3-glucoside, about 50% delphinidin-3-rutinoside and about 50% w/w delphinidin-3-glucoside, about 66.6% delphinidin-3-rutinoside and about 33.3% w/w w/w delphinidin-3-glucoside, about 25% delphinidin-3-rutinoside and about 75% w/w delphinidin-3-glucoside, and about 90% delphinidin-3-rutinoside and about 10% w/w delphinidin-3-glucoside.

The absolute anthocyanin content of the black currant berry product is not particularly limited. For example, the black currant berry product may comprise anthocyanins in an amount from about 0.1% w/w to about 100% w/w, based on the weight of the black currant berry product, including from about 20% to about 50% w/w, including about 35% w/w, based on the weight of the black currant berry product. This includes about 0.1%, 0.5%, 1%, 1.5%, 5%, 10%, 20%, 30%, 35%, 40%, 50%, 60%, 70%, 75%, 80%, 85%, 90%, 95%, 99%, or 100% w/w, or any value therebetween, of anthocyanins based on the weight of the black currant berry product.

The composition may comprise the black currant berry product along with other components suitable for oral administration. For example, the composition may comprise the black currant berry product in an amount from about 1% w/w to about 100% w/w, based on the weight of the composition, including from about 25% to about 100%, including 1%, 5%, 10%, 20%, 30%, 35%, 40%, 50%, 60%, 70%, 75%, 80%, 85%, 90%, 95%, 99%, or 100% w/w, or any value therebetween. Other components suitable for oral administration that may be present in the composition include components suitable for oral administration typically used in solid or liquid compositions intended for oral consumption. Non-limiting examples of such components include sweeteners (e.g., sugars, sugar alcohols, polyols, glycine, stevia, monk fruit, other non-nutritive sweeteners, etc.), flow agents (e.g., rice flour, silica, cellulose, cream of tartar, calcium silicate, silicon dioxide, microcrystalline cellulose, tricalcium phosphate, etc.), carriers and diluents (e.g., starches, maltodextrins, etc.), lubricants (e.g., talc, magnesium stearate, calcium stearate, colloidal silica, stearic acid, etc.) flavoring agents, coloring agents, and the like. Additionally or alternatively, the composition may be a solid composition provided with a film coat (e.g., a coating made from a material such as, but not limited to, gelatin or cellulose). The black currant berry product may be the sole physiologically active agent present in the composition. In some embodiments, the composition consists of the black currant berry product.

The compositions disclosed herein may be solid compositions or liquid compositions. In some embodiments, the composition is a solid composition in the form of a capsule suitable for oral administration. In some embodiments, the composition is a solid composition in the form of a tablet suitable for oral administration. In some embodiments, the composition is a solid composition in the form of a tablet formulated to be dissolved in water (or another orally acceptable aqueous vehicle, such as juice) prior to consumption. In some embodiments, the composition is a solid composition in the form of a powder suitable for oral administration. In some embodiments, the composition is a solid composition in the form of a powder formulated to be dissolved in water (or another orally acceptable aqueous vehicle, such as juice) prior to consumption. In some embodiments, the composition is a liquid composition in the form of a liquid concentrate suitable for oral administration. In some embodiments, the composition is a liquid composition in the form of a liquid concentrate formulated to be diluted in water (or another orally acceptable aqueous vehicle, such as juice) prior to consumption. In some embodiments, the composition is a liquid composition in the form of a beverage.

### Methods for Enhancing Sleep Quality

As noted above, the present invention relates to the surprising discovery that oral administration of compositions comprising black currant berry product as disclosed herein can provide enhanced sleep quality. Thus, disclosed herein are uses of the compositions disclosed herein in for enhancing sleep quality in a human subject in need thereof, comprising orally administering to the subject an effective amount of a composition as described herein. Also disclosed herein are compositions as described herein for use in providing enhanced sleep quality in a human subject, and uses of the compositions described herein in the preparation of medicaments for providing enhanced sleep quality. The human subject may be an adult human subject. The human subject may be a human child.

The enhanced sleep quality may be assessed with reference to various factors, including increased rapid eye movement (REM) sleep, increased deep non-REM sleep, shortened sleep latency, reduced unintended wake-ups, improved ability to fall back asleep, increased overall sleep time, and enhanced refreshed feeling upon wake-up. As used herein, “improved” or “reduced” or “shortened” refers to an improvement or reduction or shortening in an aspect of sleep quality (e.g., one or more of REM sleep, deep non-REM sleep, sleep latency, number of unintended wake-ups, ability to fall back asleep, overall sleep time, and refreshed feeling upon wake-up) after consumption of a composition as disclosed herein, as compared to prior to consumption or as compared to consumption of a similarly formulated placebo, as may be determined by conducting an assessment of the subject's sleep quality prior to and after consumption of a composition as disclosed herein, or by conducting an assessment of the subject's sleep quality after consumption of a composition as disclosed herein and after consumption of a similarly formulated placebo, or as may be demonstrated in a study conducted in other human subjects. In any embodiments, sleep quality can be assessed via a wearable device that measures one or more facets of sleep quality (e.g., an Oura Ring device, Whoop device, Fitbit device, Garmin device, etc.).Additionally or alternatively, sleep quality can be assessed via a patient diary or survey.

Thus, the methods, compositions, or uses may be effective for increasing REM sleep in a human subject. Additionally or alternatively, the methods, compositions, or uses may be effective for increasing deep non-REM sleep in a human subject. Additionally or alternatively, the methods, compositions, or uses may be effective for shortening sleep latency in a human subject. Additionally or alternatively, the methods, compositions, or uses may be effective for reducing unintended wake-ups in a human subject. Additionally or alternatively, the methods, compositions, or uses may be effective for improving ability to fall back asleep in a human subject. Additionally or alternatively, the methods, compositions, or uses may be effective for increasing overall sleep time in a human subject. Additionally or alternatively, the methods, compositions, or uses may be effective for enhancing refreshed feeling upon wake-up in a human subject.

The composition comprising black currant berry product as disclosed herein may be administered at a dose that provides any amount of the black currant berry product effective for enhancing sleep quality as described herein. For example, the composition may be administered at a dose that provides an amount of the black currant berry product of from about 1 mg to about 5000 mg. This includes doses of about 1, 5, 10, 25, 30, 40, 50, 60, 75, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1250, 1500, 1750, 2000, 2250, 2500, 2750, 3000, 3250, 3500, 3750, 4000, 4250, 4500, 4750, or 5000 mg, or any value therebetween, of black currant berry product. In some embodiments, the composition is administered at a dose that provides an amount of black currant berry product of from about 10 mg to about 800 mg, including from about 10 mg to about 400 mg. In some embodiments, the composition is administered at a dose that provides an amount of black currant berry product of from about 30 mg to about 400 mg, including from about 30 mg to about 200 mg.

Without being bound by theory, it is understood that the amount of black currant berry product effective for enhancing sleep quality may inversely vary with the anthocyanin content of the black currant berry product, such that a lower dose of a product having a higher anthocyanin content would be equivalent to a higher dose of a product having a lower anthocyanin content. In some embodiments, the composition is administered at a dose that provides an amount of black currant berry product equivalent to from about 10 mg to about 800 mg, including from about 10 mg to about 400 mg, of a black currant berry product having an anthocyanin content of about 35% w/w, based on the total weight of the black currant berry product, wherein “equivalent to” indicates about the same anthocyanin content. In some embodiments, the composition is administered at a dose that provides an amount of black currant berry product equivalent to from about 30 mg to about 400 mg, including from about 30 mg to about 200 mg, of a black currant berry product having an anthocyanin content of about 35% w/w, based on the total weight of the black currant berry product, wherein “equivalent to” indicates about the same anthocyanin content.

The composition comprising black currant berry product as disclosed herein may be administered at any time of day effective for enhancing sleep quality as described herein. For example, the methods or uses may comprise administering a composition as disclosed herein from about 4 hours before bedtime to about bedtime. In some embodiments, the methods or uses comprise administering a composition as disclosed herein at bedtime. In some embodiments, the methods or uses comprise administering a composition as disclosed herein about 30 minutes before bedtime. In some embodiments, the methods or uses comprise administering a composition as disclosed herein about 1 hour before bedtime. In some embodiments, the methods or uses comprise administering a composition as disclosed herein about 2 hours before bedtime. In some embodiments, the methods or uses comprise administering a composition as disclosed herein about 3 hours before bedtime. In some embodiments, the methods or uses comprise administering a composition as disclosed herein about 4 hours before bedtime. The composition may be administered once per day, or more than once per day. The timing and number of daily administrations may be tailored to and adjusted in view of the subject's sleep schedule.

In some aspects, the methods or uses further comprise administering one or more additional physiologically active agents. The one or more additional physiologically active agents may be administered simultaneously with the black currant berry composition. Additionally or alternatively, one or more additional physiologically active agents may be administered at a different time of day than the black currant berry composition. The one or more additional physiologically active agents may be selected from one or more of L-theanine, glycine, melatonin, gamma-aminobutyric acid, and magnesium L-threonate. Thus, in some embodiments, the one or more additional physiologically active agents is L-theanine. In some embodiments, the one or more additional physiologically active agents is glycine. In some embodiments, the one or more additional physiologically active agents is melatonin. Thus, in some embodiments, the one or more additional physiologically active agents is gamma-aminobutyric acid. In some embodiments, the one or more additional physiologically active agents is magnesium L-threonate. In some embodiments, the one or more additional physiologically active agents are two or more selected from L-theanine, glycine, melatonin, gamma-aminobutyric acid, and magnesium L-threonate. In some embodiments, the one or more additional physiologically active agents are three or more selected from L-theanine, glycine, melatonin, gamma-aminobutyric acid, and magnesium L-threonate. In some embodiments, the one or more additional physiologically active agents are each of L-theanine, glycine, melatonin, gamma-aminobutyric acid, and magnesium L-threonate.

For example, the methods or uses may further comprise administering from about 50 mg to about 800 mg of L-theanine, including from about 50 mg to about 400 mg of L-theanine. This includes about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, or 800 mg, or any value therebetween, of L-theanine.

Additionally or alternatively, the methods or uses may further comprise administering from about 500 mg to about 8000 mg of glycine, including from about 500 mg to about 5000 mg of glycine. This includes about 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, and 8000 mg, or any value therebetween, of glycine.

Additionally or alternatively, the methods or uses may further comprise administering from about 0.1 mg to about 10 mg of melatonin. This includes about 0.1, 0.2, 0.3, 0.4, 0.5, 0.6, 0.7, 0.8, 0.9, 1.0, 1.1, 1.2, 1.3, 1.4. 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 mg, or any value therebetween, of melatonin.

Additionally or alternatively, the methods or uses may further comprise administering from about 100 mg to about 2000 mg gamma-aminobutyric acid. This includes about 100, 200, 300, 400, 500, 600, 700, 800, 900, 1000, 1100, 1200, 1300, 1400, 1500, 1600, 1700, 1800, 1900, or 2000 mg, or any value therebetween, of gamma-aminobutyric acid.

Additionally or alternatively, the methods or uses may further comprise administering from about 50 mg to about 2000 mg of magnesium L-threonate. This includes about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000, 1100, 1200, 1300, 1400, 1500, 1600, 1700, 1800, 1900, or 2000 mg, or any value therebetween, of magnesium L-threonate. In some embodiments, the method further comprises administering one or more of (i) from about 50 mg to about 800 mg L-theanine, (ii) from about 500 mg to about 8000 mg glycine, (iii) from about 0.1 mg to about 10 mg melatonin, (iv) from about 50 mg to about 2000 mg magnesium L-threonate, and (v) from about 100 mg to about 2000 mg gamma-aminobutyric acid. Alternatively, the methods or uses may further comprise administering one or more of (i) from about 50 mg to about 400 mg L-theanine, (ii) from about 500 mg to about 5000 mg glycine, (iii) from about 0.1 mg to about 10 mg melatonin, (iv) from about 100 mg to about 2000 mg gamma-aminobutyric acid, and (v) from about 50 mg to about 2000 mg magnesium L-threonate.

The present invention, thus generally described, will be understood more readily by reference to the following examples, which are provided by way of illustration and are not intended to be limiting of the present invention.

### EXAMPLES

### Example 1

A 36-year old man consumed 70 mg of a black currant berry extract as described herein having an anthocyanin content of 35% by weight mixed with approximately 12 oz of water one hour before sleep. The subject noticed an unexpected and strong subjective sense of improved sleep. He wore a clinically validated wearable device which documented approximately 20% greater deep non-REM sleep with administration of the black currant berry extract. This benefit disappeared on cessation of administration of the black currant berry extract and resumed with restarting administration of the black currant berry extract.

### Example 2

A 45-year old woman had recently tried many other dietary supplements marketed as remedies for sleep in an attempt to be able to sleep at least 6 hours without waking multiple times, which she had been unable to do for years. She found the supplements to be ineffective. She then began consuming 70 mg of a black currant berry extract as described herein having an anthocyanin content of 35% by weight mixed with approximately 8 oz of water one hour before sleep. Over the full 2 months of follow-up, she described regularly being able to sleep 6 or even 8 hours without waking up.

### Example 3

A 40-year old man consumed 200 mg of a black currant berry extract as described herein having an anthocyanin content of 35% by weight in a capsule form one hour before bed. From the first night, he described feeling significantly more refreshed on waking up. When he took another product which he mistakenly believed to be equivalent, these benefits went away. The benefits resumed when he again administered the black currant berry extract of the present disclosure.

### Example 4

In a retrospective analysis of 8 individuals in a product test group of a black currant berry extract as described herein having an anthocyanin content of 35% by weight, 63% reported fewer nocturnal awakenings when using between 200 and 400 mg of the black currant extract compared to before using the product. When compared to each individual's average amount of deep sleep before using the black currant berry extract, 83% of the 40 nights slept when using 200-400 mg of the black currant berry extract had more deep sleep as measured by a clinically validated wearable sleep tracker.

While certain embodiments have been illustrated and described, it should be understood that changes and modifications can be made therein in accordance with ordinary skill in the art without departing from the technology in its broader aspects.

What is claimed is:

1. A method of providing enhanced sleep quality in a human subject in need thereof, the method comprising orally administering to the subject an effective amount of a composition comprising a black currant berry product.

2. The method of claim 1, wherein the enhanced sleep quality comprises one or more selected from increased rapid eye movement (REM) sleep, increased deep non-REM sleep, shortened sleep latency, reduced unintended wake-ups, improved ability to fall back asleep, increased overall sleep time, and enhanced refreshed feeling upon wake-up.

3. The method of claim 1, wherein the black currant berry product comprises black currant berry extract, black currant puree, black currant juice, black currant syrup, or a combination of two or more thereof.

4. The method of claim 1, wherein the black currant berry product comprises one or more selected from anthocyanins and phenolic compounds.

5. The method of claim 4, wherein the black currant berry product comprises anthocyanins in an amount from about 20% w/w to about 50% w/w based on the weight of the black currant berry product.

6. The method of claim 4, wherein the black currant berry product comprises one or more anthocyanins selected from delphinidin-3-glucoside, delphinidin-3-rutinoside, cyanidin-3-rutinoside, cyanidin-3-glucoside, cyanidin-3-O-glucoside, cyanidin-3-glucosylrutinoside, cyanidin-3-sophoroside, and peonidin-3-glucoside.

7. The method of claim 4, wherein the black currant berry product comprises one or both of delphinidin-3-glucoside and delphinidin-3-rutinoside.

8. The method of claim 4, wherein the black currant berry product comprises from about 30% w/w to about 50% w/w delphinidin-3-rutinoside, from about 4% w/w to about 25% w/w delphinidin-3-glucoside, from about 25% w/w to about 45% w/w cyanidin-3-rutinoside, and from about 2% w/w to about 10% w/w cyanidin-3-glucoside, all based on the total weight of anthocyanins present in the black currant berry product.

9. The method of claim 4, wherein the black currant berry product further comprises one or more proanthocyanidin compounds.

10. The method of claim 1, wherein the black currant berry product is administered at a dose of from about 1 mg to about 5000 mg.

11. The method of claim 1, wherein the composition is administered at a dose that provides an amount of black currant berry product equivalent to from about 10 mg to about 800 mg of a black currant berry product having an anthocyanin content of about 35% w/w, based on the total weight of the black currant berry product.

12. The method of claim 1, wherein the composition is administered at a dose that provides an amount of black currant berry product equivalent to from about 30 mg to about 400 mg of a black currant berry product having an anthocyanin content of about 35% w/w, based on the total weight of the black currant berry product.

13. The method of claim 1, wherein the composition is a solid composition.

14. The method of claim 13, wherein the composition is in the form of a capsule, tablet, or powder.

15. The method of claim 1, wherein the composition is a liquid composition.

16. The method of claim 15, wherein the composition is in the form of a liquid concentrate or beverage.

17. The method of claim 1, wherein the black currant berry product is the sole physiologically active agent present in the composition.

18. The method of claim 1, further comprising administering one or more additional physiologically active agents.

19. The method of claim 18, wherein the one or more additional physiologically active agents are selected from L-theanine, glycine, melatonin, gamma-aminobutyric acid, magnesium L-threonate, and any combination of two or more thereof.

20. The method of claim 19, comprising administering one or more of (i) from about 50 mg to about 800 mg L-theanine, (ii) from about 500 mg to about 8000 mg glycine, (iii) from about 0.1 mg to about 10 mg melatonin, (iv) from about 50 mg to about 2000 mg magnesium L-threonate, and (v) from about 100 mg to about 2000 mg gamma-aminobutyric acid.
